(19) United States
(12) Patent Application Publication
(10) Pub. No.: US 2025/0041497 A1
(43) Pub. Date: Feb. 6, 2025

(54) CONNECTIVITY SET FOR CONNECTING A POST-PROCESSING UNIT TO AN APHERESIS DEVICE

(71) Applicant: Terumo BCT, Inc., Lakewood, CO (US)

(72) Inventors: Dawn FALLER, Littleton, CO (US); James LADTKOW, Broomfield, CO (US); Briden STANTON, Highlands Ranch, CO (US)

(21) Appl. No.: 18/780,699

(22) Filed: Jul. 23, 2024

Related U.S. Application Data
(60) Provisional application No. 63/529,932, filed on Jul. 31, 2023.

(51) Int. Cl. A61M 1/34; A61M 39/28

(52) U.S. Cl. CPC A61M 1/3496; A61M 1/3401; A61M 39/28

(57) ABSTRACT

A medical system may include a separation device configured to separate a material received from a source into two or more components, a post-processing unit in fluid communication with the separation device and configured to isolate at least one of the two or more components, and a controller configured to manage operation of the separation device to separate the material received from the source into the two or more components, to cause the two or more components to move to the post-processing unit, and to return the isolated at least one of the two or more components to the separation device.

### CROSS-REFERENCE TO RELATED APPLICATIONS

This application claims the benefit of U.S. Provisional Application No. 63/529,932 filed on Jul. 31, 2023. The entire disclosure of the above application is incorporated herein by reference.

### FIELD

The present disclosure relates to medical systems including, or configured to receive or communicate with, post-processing units for specialized blood component identification and isolation.

### BACKGROUND

This section provides background information related to the present disclosure which is not necessarily prior art.

Plasma-based therapeutics can be useful for treating or mediating a variety of conditions, including for sepsis and neurological conditions. Plasma bound and plasma-based therapeutics often include the extraction of plasma from a subject or patient, the processing of the plasma as extracted from the subject or patient to remove one or more specific compounds or components, and the return of the adjusted or modified plasma to the subject or patient. The extraction, processing, and return is often generally referred to as targeted plasma modification or secondary plasma processing. In certain variations, plasma bound and plasma-based therapeutics may, additionally, or alternatively, include the introduction of donated and processed plasma and/or saline solution to the subject or patient. The extraction and introduction of donated and processed plasma and/or saline solution is often generally referred to as plasma exchange.

The plasma exchange removes disease antagonists but also often removes other essential components, including, for example, drugs that the subject or patient may be taking, essential proteins, and the like. The plasma exchange may expose the vulnerable subject or patient to risks associated with donated plasma. Targeted plasma modification may reduce these risks. In the instance of targeted plasma modification, a subject or patient's plasma is often passed over an adsorption or immunoadsorption column for selective removal. For example, the column may be treated with one or more ligands that are selected for binding with one or more specific compounds (e.g., proteins) such that only the specific compounds are removed. The targeted plasma modification is commonly completed using a dialysis machine; however, an apheresis system is often more efficient. To use an apheresis system for targeted plasma modification, the column may be connected to, or readily couplable to, an exchange set that fluidically connects the subject or patient and the apheresis system.

### SUMMARY

This section provides a general summary of the disclosure and is not a comprehensive disclosure of its full scope or all of its features.

In various aspects, the present disclosure provides a medical system.

In at least one example embodiment, the medical system may include a separation device configured to separate a material received from a source into two or more components, a post-processing unit in fluid communication with the separation device and configured to isolate at least one of the two or more components, and a controller configured to manage operation of the separation device to separate the material received from the source into the two or more components, to cause the two or more components to move to the post-processing unit, and to return the isolated at least one of the two or more components to the separation device.

In at least one example embodiment, the medical system may further include a tube establishing the fluid communication between the separation device and the post-processing unit.

In at least one example embodiment, the fluid communication between the separation device and the post-processing unit may be a first fluid communication between the separation device and the post-processing unit, and the medical system may further include a second fluid communication between the post-processing unit and the separation device to return the isolated at least one of the two or more components to the separation device.

In at least one example embodiment, the tube may be a first tube and the medical system may further include a second tube establishing the second fluid communication between the post-processing unit and the separation device to return the isolated at least one of the two or more components to the separation device.

In at least one example embodiment, the first and second tubes may be provided as a connectivity set.

In at least one example embodiment, the medical system may further include a third tube establishing fluid communication between the separation device and the source.

In at least one example embodiment, the first, second, and third tubes may be provided as a connectivity set.

In at least one example embodiment, the second and the third tubes may be provided as a connectivity set.

In at least one example embodiment, the post-processing unit may include a column.

In at least one example embodiment, the post-processing unit may include a filter.

In various aspects, the present disclosure provides a therapeutic system.

In at least one example embodiment, the therapeutic system may include a flow path fluidically connecting a separation device and a post-processing device, where the separation device is configured to separate a material received from a source into two or more components, and the post-processing device is configured to isolate at least one of the two or more components.

In at least one example embodiment, the flow path may be a first flow path, and the therapeutic system may further include a second flow path fluidically connecting the separation device and the post-processing device to return the isolated at least one of the two or more components to the separation device.

In at least one example embodiment, the first flow path and the second flow path may be provided as a connectivity set.

In at least one example embodiment, the therapeutic system may further include a third flow path fluidically connecting the separation device and the source.

In at least one example embodiment, the first, second, and third flow paths may be provided as a connectivity set.

In at least one example embodiment, the second and the third flow paths may be provided as a connectivity set.

In at least one example embodiment, the post-processing device may include a column.

In at least one example embodiment, the post-processing device may include a filter.

In various aspects, the present disclosure provides a connectivity set for connecting a separation device and a post-processing unit.

In at least one example embodiment, the connectivity set may include a first tube collection and a second tube collection. The first tube collection may include a first tube length having a first end and a second end, where the first end of the first tube length is couplable to the separation device, and the second end of the first tube length is couplable to the post-processing unit. The second tube collection may include a second tube length having a first end and a second end, where the first end of the second tube length is couplable to the post-processing unit, and the second end of the second tube length is couplable to the separation device. The separation device may be configured to separate a material received from a source into two or more components. The post-processing unit may be configured to isolate at least one of the two or more components.

In at least one example embodiment, the first tube collection may include one or more first clamps.

In at least one example embodiment, the second tube collection includes one or more second clamps.

In at least one example embodiment, the one or more first clamps are a first color, and the one or more second clamps are a second color, where the second color is different from the first color.

In at least one example embodiment, the connectivity set may further include a third tube collection including a third tube length couplable to the separation device and the source.

In at least one example embodiment, the third tube collection may include one or more third clamps.

In various aspects, the present disclosure provides a connectivity set for connecting a separation device and a post-processing unit.

In at least one example embodiment, the connectivity set may include a first tube length, a second tube length, a third tube length, a fourth tube length, and a fifth tube length. The first tube length may have a first end and a second end, where the first end of the first tube length may be couplable to a first bag. The second tube length may have a first end and a second end, where the first end of the second tube length may be couplable to the second end of the first tube length, and the second end of the second tube length may be couplable to a first end of the post-processing unit. The third tube length may have a first end and a second end, where the first end of the third tube length may be couplable to the second end of the first tube length. The fourth tube length may have a first end and a second end, where the first end of the fourth tube length may be couplable to the second end of the third tube length, and the second end of the fourth tube length may be couplable to a second bag. The fifth tube length may have a first end and a second end, where the first end of the fifth tube length may be couplable to a second end of the post-processing unit, and the second end of the fifth tube length may be couplable to one or more other components of the separation device.

In at least one example embodiment, the first tube length, the second tube length, and the third tube length may be joined and provided as a first distinct tube collection.

In at least one example embodiment, the fourth tube may be provided as a second distinct tube collection.

In at least one example embodiment, the fifth tube may be provided as a third distinct tube collection.

In at least one example embodiment, at least one of the first tube length, the second tube length, the third tube length, the fourth tube length, and the fifth tube length may include one or more clamps.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is an illustration of an example apheresis device in accordance with at least one example embodiment of the present disclosure;

FIG. 2 is an illustration of an example tubing and bag set for use in or with apheresis devices, like the example apheresis device illustrated in FIG. 1, in accordance with at least one example embodiment of the present disclosure;

FIG. 3 is an illustration of an example disposable set for use in or with medical systems, like the example apheresis device illustrated in FIG. 1, in accordance with at least one example embodiment of the present disclosure;

FIG. 4 is an illustration of an example tubing and bag set for aligning a post-processing unit for use in or with medical systems, like the example apheresis device illustrated in FIG. 1, in accordance with at least one example embodiment of the present disclosure; and

FIG. 5 is an illustration of the example tubing and bag set as illustrated in FIG. 4, where the example tubing and bag set includes the post-processing unit and includes one or more further tube lengths for aligning the post-processing unit for use in or with apheresis devices, like the example apheresis device illustrated in FIG. 1, in accordance with at least one example embodiment of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Various components are referred to herein as “operably associated.” As used herein, “operably associated” refers to components that are linked together in operable fashion and encompasses embodiments in which components are linked directly, as well as embodiments in which additional components are placed between the linked components. “Operably associated” components can be “fluidly associated.” “Fluidly associated” refers to components that are linked together such that fluid can be transported between them. “Fluidly associated” encompasses embodiments in which additional components are disposed between the two fluidly associated components, as well as components that are directly connected. Fluidly associated components can include components that do not contact fluid but contact other components to manipulate the system (e.g., a peristaltic pump that pumps fluids through flexible tubing by compressing the exterior of the tube).

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some example embodiments, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

Example embodiments will now be described more fully with reference to the accompanying drawings.

The present disclosure relates to medical systems including, or configured to receive or communicate with, post-processing units for specialized blood component identification and isolation. In at least one example embodiment, the medical systems may include apheresis devices like those detailed in U.S. Pat. No. 8,123,713, titled SYSTEM AND METHODS FOR COLLECTING PLASMA PROTEIN FRACTIONS FROM SEPARATED BLOOD COMPONENTS and issued Feb. 28, 2012 and/or U.S. Pat. No. 7,780,618, titled EXTRACORPOREAL BLOOD PROCESSING APPARATUS AND METHODS WITH PRESSURE SENSING and issued Aug. 24, 2010 and/or U.S. Pat. No. 10,704,023, titled SEPARATING COMPOSITE LIQUIDS and issued Jul. 7, 2020 and/or U.S. Pat. No. 9,435,736, titled SYSTEM FOR BLOOD SEPARATION WITH SHIELDED EXTRACTION PORT AND OPTICAL CONTROL and issued Sep. 6, 2016 and/or U.S. Pat. No. 8,057,376, titled BLOOD PROCESSING APPARATUS WITH SEALED DIFFUSER IN OPTICAL CONTROL APPARATUS and issued Nov. 15, 2011, the entire disclosures of which are hereby incorporated by references.

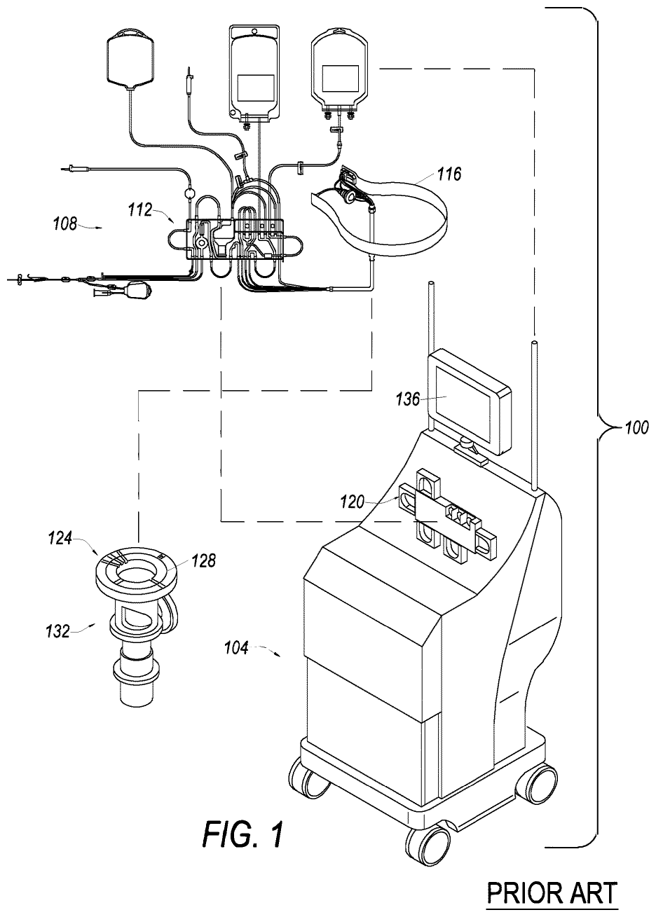

Apheresis devices often include one or more connections configured to move whole blood and/or blood components to and from blood component separation devices. Blood component separation devices may be housed within the apheresis devices and often includes centrifuges. By way of example, FIG. 1 is an illustration of an example apheresis device 100. The apheresis device 100 may be used for whole blood separation processes, where whole blood is withdrawn form a donor or a subject or a patient or source and provided to a blood component separation device 104 where the whole blood is separated into various compounds, components, or elements some of which are collected and a remainder is returned to the donor or the subject or the patient or the source.

In at least one example embodiment, the whole blood may be withdrawn from the donor or the subject or the patient or the source and directed through a bag and tubing set 108, which is configured to adapted to be received by (e.g., mounted in) the blood component separation device 104. The bag and tubing set 108 may include, for example, an extracorporeal tubing circuit 112 and a processing vessel 116, which together define a closed, sterile, and disposable system. In at least one example embodiment, the blood component separation device 104 may include a pump/valve/sensor assembly 120 that interfaces with the extracorporeal tubing circuit 112. In at least one example embodiment, the blood component separation device 104 may include a centrifuge assembly 124 that interfaces with the processing vessel 116. For example, the centrifuge assembly 124 may include a channel 128 in a rotatable rotor assembly 132, where the processing vessel 116 may be fitted within the channel 128 and the rotatable rotor assembly 132 is configured to provide the centrifugal forces required to separate the whole blood into its one or more compounds or components or elements.

In at least one example embodiment, the whole blood may flow from the donor or the subject or the patient or the source through the extracorporeal tubing circuit 112 and into the rotating processing vessel 116. Within the processing vessel 116, the whole blood may be separated into the one or more compounds or components or elements and at least one of the one or more separated compounds or components or elements may be removed from the processing vessel 116. For example, the one or more separated compounds or components or elements that are not being retained for collection (for example, for therapeutic treatment) may be removed from the processing vessel 116 and returned to the donor or the subject or the patient or the source via the extracorporeal tubing circuit 112. In at least one example embodiment, the flow of the whole blood may be substantially continuous. In various other example embodiments, the collection process using the example apheresis device 100 may include batch processes (e.g., non-continuous inflow of whole blood and/or non-continuous outflow of separated blood components) or smaller scale batch or continuous red blood cell/plasma separation systems, with or without blood compounds or components or elements being returned to the donor or the subject or the patient or the source.

The apheresis device 100 may include one or more processors that are configured to control the operations of the blood component separation device 104. The one or more processors may be part of a computer system. The computer system may include components or parts that allow a user or operator of the apheresis device 100 to interface with the computer system. For example, the apheresis device 100 may include memory and storage devices (such as RAM, ROM (e.g., CD-ROM, DVD), magnetic drives, optical drives, flash memory, and the like); communication/networking devices (e.g., wired (such as modems/network cards) and/or wireless (such as Wi-Fi)); input devices such keyboard(s), touch screen(s), camera(s), and/or microphone(s); and/or output device(s) such as display(s), and audio system(s). In at least one example embodiment, the apheresis device 100 may include a graphical user interface 136 with a display that includes, for example, an interactive touch screen

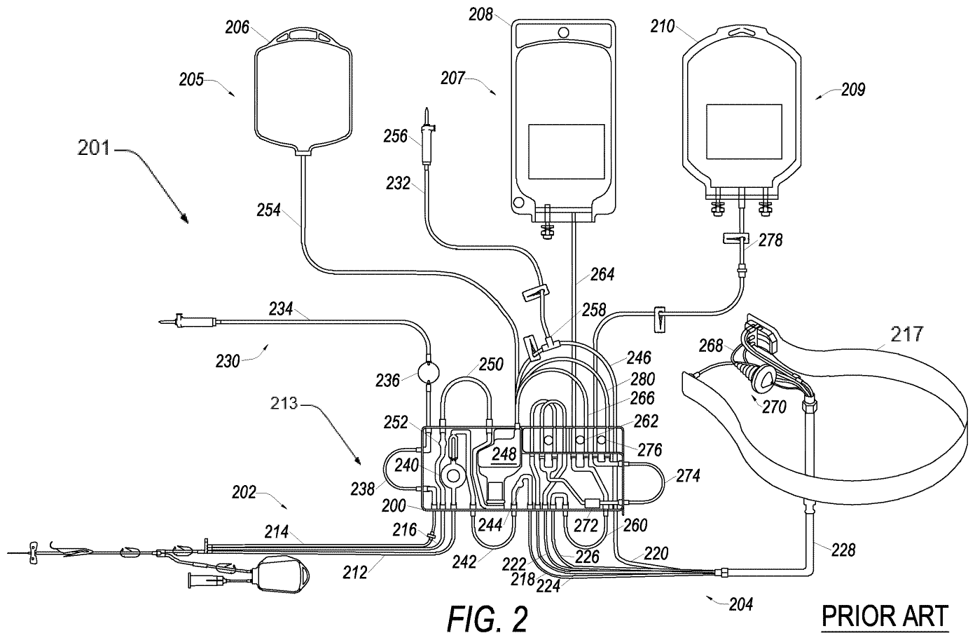

FIG. 2 is an illustration of an example tubing and bag set (or exchange set) 201 for use in or with apheresis devices, like the example apheresis device 100 illustrated in FIG. 1. For example, the tubing and bag set 201 illustrated in FIG. 2 may be used as the bag and tubing set 108 for the apheresis device 100 illustrated in FIG. 1. Like the bag and tubing set 108 illustrated in FIG. 1, the tubing and bag set 201 illustrated in FIG. 2 may include an extracorporeal tubing circuit 213, where the extracorporeal tubing circuit 213 includes a cassette 200 and a number of tubing assemblies (or collection assemblies or tubing segments) 202, 204, 205, 207, and 209.

In at least one example embodiment, the tubing assemblies may include a blood removal-return tubing assembly 202 that provides, for example, one or more lines 212, 214, 216 for removal of whole blood from the donor or the subject or the patient or the source and introducing the whole blood to the remainder of the extracorporeal tubing circuit 213 and also returning blood compounds or components or elements or other fluids to the donor or the subject or the patient or the source. For example, the blood removal-return tubing assembly 202 may include a needle interface for withdrawing the whole blood from the donor or the subject or the patient and introducing the whole blood to the remainder of the extracorporeal tubing circuit 213 and also returning blood compounds or components or elements or other fluids to the donor or the subject or the patient or the source. Although a single needle configuration is illustrated, it should be appreciated that, in various other example embodiments, other configuration may be appropriate, including, for example, a double needle interface may be used.

In at least one example embodiment, the tubing assemblies may include a blood inlet tubing assembly (or blood component tubing assembly) 204. The cassette 200 may be disposed between the blood removal-return tubing assembly 202 and the blood inlet tubing assembly 204, where the blood inlet tubing assembly 204 provides an interface between the cassette 200 and a processing vessel 217. The processing vessel 217 may be the same as the processing vessel 116 illustrated in FIG. 1. Similar to the extracorporeal tubing circuit 112 and processing vessel 116 of FIG. 1, the extracorporeal tubing circuit 213 and the processing vessel 217 of FIG. 2 may be pre-connected to form a closed, sterilized, disposable assembly for a single use.

The blood inlet tubing assembly 204 may include one or more tubing lines (or tubing segments) 218, 220, 222, 224, and 226 that are configured for transporting the whole blood and/or one or more components to and from the processing vessel 217. For example, in at least one example embodiment, as illustrated, the blood inlet tubing assembly 204 may include five tubing segments. The cassette 200 may be configured orient one or more of the different tubing assemblies within predetermined space relationships for ultimate engagement with other components, including, for example, different valve members, of the tubing and bag set 201 and/or apheresis device. In at least one example embodiment, the cassette 200 may be connected, or couplable to, an anticoagulant tubing assembly 230, a vent bag 206, a plasma collection bag 208, a white blood cell collection bag 210, or any combination thereof.

When the extracorporeal tubing circuit 213 is mounted on a blood component separation device, like the blood component separation device 104 illustrated in FIG. 1, a saline solution may be used to prime the extracorporeal tubing circuit 213, for example, through a line 234. In at least one example embodiment, line 234 may include a filter 236. The saline solution may flow through an internal passageway in the cassette 200 and through anticoagulant line 214 to the distal end of the blood removal-return assembly 202. The saline solution may then flow up a blood removal line 212 into the other tubes and passageways of the extracorporeal tubing circuit 213 during preparation for blood processing. After preparation, a supply or bag (not shown) of anticoagulant may be connected to a distal end of the anticoagulant tubing assembly 230 in place of a supply or bag of the saline solution. The anticoagulant solution may flow past the filter 236 and a first pump loop 238 through the anticoagulant line 214 to the distal end of the blood removal assembly 202. The pump loop 238, as well as other pump loops, may be couple with peristaltic pumps on the blood component separation device, while the blood component separation device controls the direction and rate of flow of the fluids by controlling the speed and direction of the peristaltic pumps and the position of various valves.

The blood removal line 212 may conduct the whole blood into the cassette 200, where the whole blood passes a first pressure sensor 240 and a second pump loop 242. A second pressure sensor 244 may be disposed between second pump loop 242 with its associated pump and blood inflow line 218 to the processing vessel 217 and may be configured to sense the fluid pressure effective at an inlet to the processing vessel 217. A red blood cell outlet tubing line 220 of the blood component tubing assembly 204 may extend from the processing vessel 217. The red blood cell outlet tubing line 220 connects to a return loop 246 to a return reservoir 248. The return reservoir 248 may contact sensors on the blood component separation device that detect low and high fluid levels. The blood component separation device may keep the fluid in the reservoir 248 between these two levels by controlling flow out of the reservoir past a return pump loop 250 and a return pressure sensor 252. A vent bag 206 may be connected to the reservoir 248 through a vent tube 254 and air may flow between the reservoir 248 and the vent bag 206 in a sterile manner. Fluid may flow into a return line 216 in the blood removal-return assembly 202. The blood removal-return assembly 202 may include the anticoagulant line 214 for priming or anti-coagulant. If desired, red blood cells could be withdrawn through replacement line 232 and collected in a collection bag (not shown). When desired, plasma may be withdrawn from the processing vessel 217 through plasma line 226 to a pump loop 260. A valve 262 may divert the plasma either into a collect tube 264 to the plasma collection bag 208 or into connecting loop or line 266 to the reservoir 248. Excess plasma in the reservoir 248 may be returned to the donor or the subject or the patient or the source in the same way as red blood cells. A bag containing replacement fluid (not shown) may be connected to a spike or Luer connector 256 on the replacement line 232, allowing replacement fluid to pass through the return loop 246 into the reservoir 248. In at least one example embodiment, replacement line 232 may be connected to return loop 246 through a junction 258 and manual closures or clamps may be provided to direct the flow of blood compounds or components or elements and/or replacement fluid.

White blood cells and platelets may flow out of the processing vessel 217 through a cell line 268 into a cell separation chamber 270. The contents of the cell separation chamber 270 may flow out of the separation chamber through an outlet. In the cassette 200, the fluid from the cell separation chamber 270 may pass a red-green photo sensor 272, which may be used to control periodic flushing of white blood cells out of the cell separation chamber 270 into the white blood cell collection bag 210. The selected cells may flow through a pump loop or common line 274, which engages a peristaltic pump on the blood component separation device. The pump loop 274 may connect to a valved passageway in the cassette 200. The blood component separation device may control a valve 276 to direct white blood cells or other selected cells either into a collect tube 278 and thence into the white blood cell collection bag 210 or into a connection loop or line 280 and thence into the reservoir 248. For platelet collection, excess white blood cells in the reservoir 248 may be returned to the donor or the subject or the patient or the source in the same way as red blood cells and plasma. Alternatively, for mesenchymal stem cell (MNC) collection, where platelets are usually returned to the donor or the subject or the patient or the source, the mesenchymal stem cell may be withdrawn into the collect tube 278 for storage in the white blood cell collection bag 210.

During a blood removal, whole blood may be passed from the donor or the subject or the patient or the source into the blood removal line 212 of blood removal tubing assembly 202. The blood may be pumped by the blood component separation device, for example, via pump loop 242, to the processing vessel 217 via the cassette 200 and blood inflow line 218 of the blood component tubing assembly 204. Separation processing may then occur on a substantially continuous basis in the processing vessel 217 (i.e., blood flows substantially continuously therein) may be continuously separated and may flow as separated components there from. After separation processing in processing vessel 217, uncollected blood components may be transferred from the processing vessel 217 to and through cassette 200 and into reservoir 248 of cassette 200, which may be filled up to a predetermined level. The blood component separation device may initiate a blood return sub-mode where components may be returned to the donor or the subject or the patient or the source through return line 216. The cycle between blood removal and blood return sub-modes may continue until a predetermined amount of blood components have been harvested.

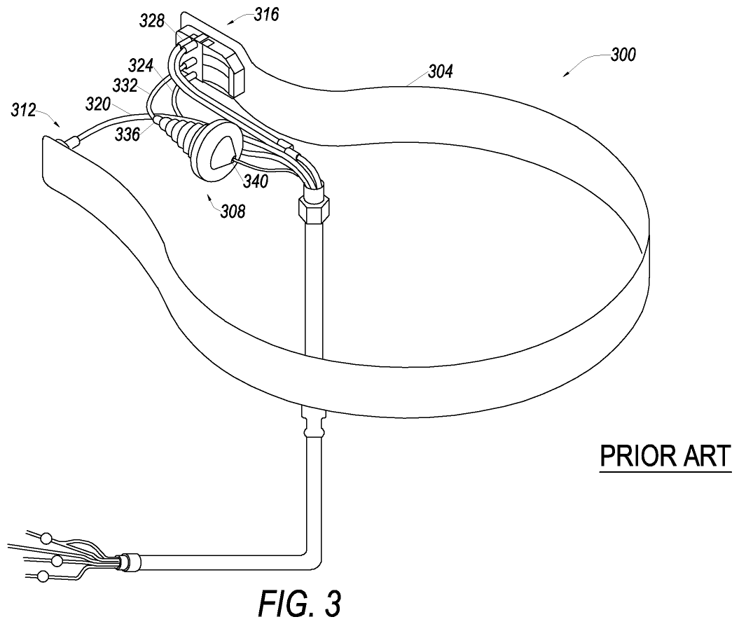

FIG. 3 is an illustration of an example disposable set 300 for use in or with apheresis systems, like the example apheresis device 100 illustrated in FIG. 1. The disposable set 300 includes a processing vessel 304 and a separation chamber 308. In at least one example embodiment, the disposable set 300 may be used to provide a blood processing disposable set, in which processing vessel 304 may be used as the processing vessel 116 as illustrated in FIG. 1, and the separation chamber 308 may be used as cell separation chamber 270 as illustrated in FIG. 2.

The processing vessel 304 may have a generally annular flow path that includes an inlet portion 312 and an opposing outlet portion 316. An inflow tube 320 may connect to the inlet portion 312 and may be configured to convey a fluid into the processing vessel 304 for separating into components (e.g., whole blood into blood components). In at least one example embodiment, the disposable set 300 may be used in a centrifuge that rotates processing vessel 304, and as a result of centrifugal forces, separates liquids into one or more components. In such instances, substances (e.g., whole blood) entering the inlet portion 312, may flow around the processing vessel 304 and stratify according to differences in density in response to rotation of the centrifuge. The outlet portion 316 may include outlets for the components of the separated liquid.

When the composite liquid being separated includes whole blood, line 324 may be used as a red blood cell line, line 328 may be used as a plasma line, and line 332 may be used as the buffy coat or white blood cell line for removing the separated components from the processing vessel 304. Each of the components separated in the processing vessel 304 may be collected and removed in only one area of the processing vessel 304, namely the outlet portion 316.

In at least one example embodiments, a component separated in the processing vessel 304 may be further processed to separate additional components. In such instances, the outlet of the line 332 may be connected to the separation chamber inlet 336 to transfer intermediate density components to chamber 308. When the liquid being separated includes whole blood, the portion transferred into chamber 308 may include the buffy coat. The buffy coat may include white blood cells or mesenchymal stem cells may be transferred into separation chamber 308. Components initially separated in the processing vessel 304 may be further separated in the cell separation chamber 308. For example, white blood cells could be separated from plasma and platelets in the buffy coat using the cell separation chamber 308. This further separation may take place, for example, by forming a saturated fluidized bed of particles in the cell separation chamber 308. Plasma and platelets may flow out of the cell separation chamber 308 through outlet 340. White blood cells or mesenchymal stem cells may be retained in the chamber 308. In other example embodiments, other components may be separated including, for example, granulocytes from a red blood cell component.

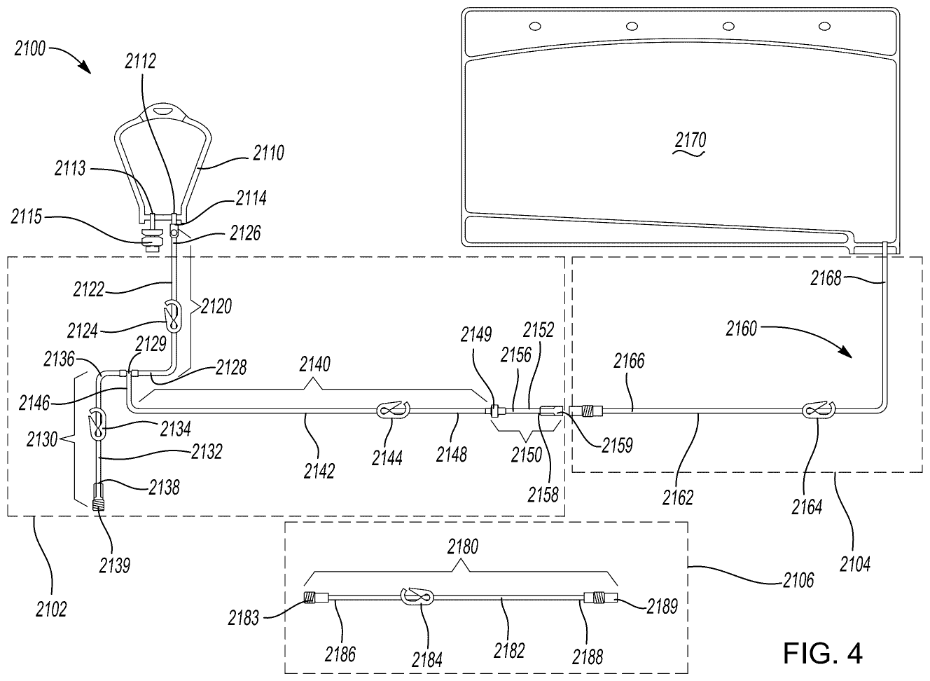

FIG. 4 is an illustration of an example tubing and bag set (or connectivity sets) 2100 for aligning a post-processing unit (or a post-processing component or post-processing device) for use in or with apheresis devices, like the example apheresis system illustrated in FIG. 1. The post-processing unit may include a filter, an adsorption column, or a combination of the filter and the adsorption column. The filter further identifies and isolates compounds or components or elements of the fluid based on a greater than or less than of a specific size and the adsorption column identifies and isolates compounds or components or elements based in response to a specific sizing range. That is, the filter will identify and isolate all compounds or components or elements that are greater than a certain filter dimension, or alternatively all compounds or components or elements that are less than a certain filter dimension, while the adsorption column will identify and isolate only those compounds or components or elements having a specific size and not larger or smaller compounds or components or elements.

The tubing and bag set 2100 may be configured to fluidically associate (e.g., connect) the post-processing unit and apheresis devices, such as the apheresis system illustrated in FIG. 1. In at least one example embodiment, the tubing and bag set 2100 may be couplable to another tubing and bag set that connects the donor or the subject or the patient or the source and a respective apheresis device. For example, in at least one example embodiment, ends 2138 and 2186 of the tubing and bag set 2100 may be couplable to opposing sides of the post-processing unit and end 2188 of the tubing and bag set 2100 may be couplable to or along the red blood cell outlet tubing line 220 of the tubing and bag set 201 illustrated in FIG. 2. During use, line 234 of the (first) tubing and bag set 201 may be closed (for example, using one or more valves or clamps) to divert plasma (and/or other compounds or components or elements) as collected from the donor or the subject or the patient or the source to the red blood cell outlet tubing line 220 and to end 2188 of the (second) tubing and bag set 2100, where the tubing and bag set 2100, as further detailed below, directs the collected plasma (and/or other compounds or components or elements) to a post-processing unit. The tubing and bag set 2100 in turn may be configured so that effluent from the post-processing unit is directed to a waste bag 2170 during priming and/or flushing of the post-processing unit or to a buffer or plasma bag 2110 so to be returned to the donor or the subject or the patient or the source. The buffer bag 2110 may be configured to compensate for any imbalance in pump flow rates and assures that the modified or treated component (e.g., plasma) is always available to be returned to the donor or the subject or the patient or the source. For example, the buffer bag 2110 may provide a free surface for the fluid, for example, to prevent pressurization or vacuum generation due to imbalances in flow. The buffer bag 2110 may be configured to provide a consistent pressure head for downstream pumps.

With renewed reference to FIG. 4, in at least one example embodiment, the tubing and bag set 2100 fluidically associating the post-processing unit and apheresis devices may include a first collection 2102 of one or more adjoining tube sections, a second collection 2104 of one or more adjoining tube sections, and also a third collection 2106 of one or more adjoining tube sections. The first collection 2102 may include one or more tubes connected to the buffer bag 2110. The second collection 2104 may include one or more tubes connected to the waste bag 2170. In at least one example embodiment, the post-processing unit may be disposed between the first collection 2102 and the third collection 2106 and the third collection 2106 may be couplable to the apheresis system. More particularly, the third collection 2106 may be couplable to another tubing and bag set (e.g., tubing and bag set 201 as illustrated in FIG. 2) that is itself couplable to the apheresis system.

In at least one example embodiment, the first collection 2102 may be provided as a first assembled collection, the second collection 2104 may be provided as a second assembled collection, and the third collection 2106 may be provided as a third assembled collection. In at least one example embodiment, the first, second, and third assembled collections may be provided as distinct components during storage and shipping of the tubing and bag set 2100. In at least one example embodiment, the first collection 2102 and the second collection 2104 may be provided as a first assembled collection and the third collection 2106 may be provided as a second assembled collection. In at least one example embodiment, the first and second assembled collection may be provided as distinct components during storage and shipping of the tubing and bag set 2100. In each instance, the first collection 2102 may include a first tube section 2120 that may be couplable to a second tube section 2130 and a third tube section 2140. The first collection 2102 may include a fourth tube section 2150 that may be couplable to the third tube section 2140 away from the first tube section 2120.

The first tube section 2120 may include a first tube length 2122 having a first end 2126 and a second end 2128. In at least one example embodiment, the first tube section 2120 may be referred to as a plasma line. Although a single tube length is illustrated, it should be appreciated that, in various other example embodiments, the first tube section 2120 may be defined by two or more tube lengths or sections including one or more clamps (like the first clamp 2124) and/or one or more couplings configured, for example, to join the two or more tube lengths or sections.

The first tube section 2120 may include a first clamp 2124 that may be positioned at a point along the first tube length 2122 between the first and second ends 2126, 2128. The first clamp 2124 may be movable between a first or open position and/or a second or intermediate position and/or a third or close position. In the first position, the first clamp 2124 may apply no or minimal pressure to the first tube length 2122 and fluid may flow freely therethrough. In the intermediate position, the first clamp 2124 may apply a closing pressure to the first tube length 2122 to at least partially obscure fluid flow therethrough. In the close position, the first clamp 2124 may apply a closing pressure to the first tube length 2122 to completely obscure fluid flow therethrough. The first clamp 2124 may be positioned at any point along the first tube length 2122 between the first and second ends 2126, 2128. For example, in at least one example embodiment, the first clamp 2124 may be disposed about halfway along the first tube length 2122 between the first and second ends 2126, 2128. In various other example embodiments, the first clamp 2124 may be disposed along the first tube length 2122 closer or nearer to the first end 2126 than the second end 2128. In still other example embodiments, the first clamp 2124 may be disposed along the first tube length 2122 closer or near to the second end 2128 than the first end 2126.

The first end 2126 of the first tube length 2122 may be couplable to the buffer bag 2110 that receives the modified plasma. In at least one example embodiment, the buffer bag 2110 may include a first port or opening 2112 and a first coupling 2114 configured to join together the first end 2126 of the first tube length 2122 and the first opening 2112. Although the buffer bag 2110 is described as including the first coupling 2114, it should be appreciated that, in various example embodiments, the first coupling 2114 may be provided with the first collection 2120. Although not illustrated, it should be appreciated that, in various example embodiments, the first end 2126 of the first tube length 2122 may extend into the buffer bag 2110. Although not illustrated, it should be appreciated that, in various example embodiments, the first end 2126 of the first tube length 2122 may be integrally formed with the buffer bag 2110. In each instance, the buffer bag 2110 may include a second port or opening 2113 and optionally a second coupling 2115 that may be couplable to the apheresis device 100 (for example, via one or more other tube lengths 2252, 2254, 2256) so as to be able to return the modified fluid (e.g., plasma) to the donor or the subject or the patient or the source. In at least one example embodiment, the second port 2113 may be configured to receive a spike connector that is, for example, another tubing and bag set (e.g., tubing and bag set 201 as illustrated in FIG. 2) that connects the donor or the subject or the patient or the source and the apheresis system.

The second end 2128 of the first tube length 2122 may be couplable to the second tube section 2120 and the third tube section 2140. For example, a third coupling 2129 may be configured to join together the second end 2128 of the first tube length 2122 to the second tube section 2120 and the third tube section 2140. More specifically, the third coupling 2129 may join together the second end 2128 of the first tube length 2122 and a first end 2136 of the second tube section 2120 and also the second end 2128 of the first tube length 2122 and a first end 2146 of the third tube section 2140. In at least one example embodiment, the third coupling 2129 may include a t-coupling.

The second tube section 2130 may include a second tube length 2132 having a first end 2136 and a second end 2138. In at least one example embodiment, the second tube section 2130 may be referred to as an outlet line. Although a single tube length is illustrated, it should be appreciated that, in various other example embodiments, the second tube section 2130 may be defined by two or more tube lengths or sections including one or more clamps (like the second clamp 2134) and/or one or more couplings configured, for example, to join the two or more tube lengths or sections.

The first end 2136 of the second tube length 2132 may be couplable to the first tube length 2122, for example, via the third coupling 2129. The second end 2138 of the second tube length 2132 may be couplable to the processing unit. More particularly, the second end 2138 of the second tube length 2132 may be couplable to a first end of the post-processing unit, for example, via a fourth coupling 2139. In at least one example embodiment, the second tube section 2130 (and more particularly, the second end 2138 of the second tube length 2132) may include at least a portion of the fourth coupling 2139.

The second tube section 2130 may include a second clamp 2134 that may be positioned at a point along the second tube length 2132 between the first and second ends 2136, 2138. Like the first clamp 2124, the second clamp 2134 may be movable between a first or open position and/or a second or intermediate position and/or a third or close position. In the first position, the second clamp 2134 may apply no or minimal pressure to the second tube length 2132 and fluid flows freely therethrough. In the intermediate position, the second clamp 2134 may apply a closing pressure to the second tube length 2132 to at least partially obscure fluid flow therethrough. In the close position, the second clamp 2134 may apply a closing pressure to the second tube length 2132 to completely obscure fluid flow therethrough. The second clamp 2134 may be positioned at any point along the second tube length 2132 between the first and second ends 2136, 2138. For example, in at least one example embodiment, the second clamp 2134 may be disposed about halfway along the second tube length 2132 between the first and second ends 2136, 2138. In various other example embodiments, the second clamp 2134 may be disposed along the second tube length 2132 closer to the first end 2136 than the second end 2138. In still other example embodiments, the second clamp 2134 may be disposed along the second tube length 2132 closer to the second end 2138 than the first end 2136.

The third tube section 2140 may include a third tube length 2142 having a first end 2146 and a second end 2148. In at least one example embodiment, the third tube section 2140 may be referred to as an outlet line. Although a single tube length is illustrated, it should be appreciated that, in various other example embodiments, the second tube section 2130 may be defined by two or more tube lengths or sections including one or more clamps (like the third clamp 2144) and/or one or more couplings configured, for example, to join the two or more tube lengths or sections.

The first end 2146 of the third tube length 2142 may be couplable to the first tube length 2122, for example, via the second coupling 2129. The second end 2148 of the third tube length 2142 may be couplable to the fourth tube section 2150. More particularly, the second end 2148 of the third tube length 2142 may be couplable to a first end 2156 of the fourth tube section 2150, for example, via a fifth coupling 2149. In at least one example embodiment, the third tube section 2140 (and more particularly, the first end 2156 of the fourth tube section 2150) may include at least a portion of the fifth coupling 2149.

The third tube section 2140 may include a third clamp 2144 positioned at a point along the third tube length 2142 between the first and second ends 2146, 2148. Like the first and second clamps 2124, 2134, the third clamp 2144 may be movable between a first or open position and/or a second or intermediate position and/or a third or close position. In the first position, the third clamp 2144 may apply no or minimal pressure to the third tube length 2142 and fluid flows freely therethrough. In the intermediate position, the third clamp 2144 may apply a closing pressure to the third tube length 2142 to at least partially obscure fluid flow therethrough. In the close position, the third clamp 2144 may apply a closing pressure to the third tube length 2142 to completely obscure fluid flow therethrough. The third clamp 2144 may be positioned at any point along the third tube length 2142 between the first and second ends 2146, 2148. For example, in at least one example embodiment, the third clamp 2144 may be disposed about halfway along the third tube length 2142 between the first and second ends 2146, 2148. In various other example embodiments, the third clamp 2144 may be disposed along the third tube length 2142 closer to the first end 2146 than the second end 2148. In still other example embodiments, the third clamp 2144 may be disposed along the third tube length 2142 closer to the second end 2148 than the first end 2146.

The fourth tube section 2150 may include a fourth tube length 2152 having a first end 2156 and a second end 2158. In at least one example embodiment, the fourth tube section 2150 together with the third tube section 2140 may be referred to as a waste line. Although two sections are illustrated, it should be appreciated that, in various other example embodiments, the third tube section 2140 and/or the fourth tube section 2150 may be formed as a single tube length to section, or alternatively still, three or more tube lengths or sections, including one or more clamps (like the third clamp 2144) and/or one or more couplings configured, for example, to join the three or more tube lengths or sections.

The first end 2156 of the fourth tube section 2150 may be couplable to the second end 2149 of the third tube length 2142, for example, via a fifth coupling 2149. In at least one example embodiment, the fourth tube section 2150 (and more particularly, the first end 2156 of the fourth tube section 2150) may include at least a portion of the fifth coupling 2149. The second end 2158 of the fourth tube section 2150 may be couplable to the second collection 2104, for example, via a sixth coupling 2159. More particularly, the second end 2158 of the fourth tube section 2150 may be couplable to a first end 2166 of a fifth tube length 2162 of the second collection 2104. In at least one example embodiment, the second end 2158 of the fourth tube section 2150 (and more particularly, the second end 2158 of the fourth tube section 2150) may include at least a portion of the sixth coupling 2159.

The second collection 2108 may include a fifth tube section 2160. The fifth tube section 2160 may include a fifth tube length 2162 having a first end 2166 and a second end 2168. In at least one example embodiment, the fifth tube section 2160 may be referred to as a waste line. Although a single tube length is illustrated, it should be appreciated that, in various other example embodiments, the fifth tube section 2160 may be defined by two or more tube lengths or sections including one or more clamps (like the fourth clamp 2164) and/or one or more couplings configured, for example, to join the two or more tube lengths or sections.

The first end 2166 of the fifth tube length 2162 may be couplable to the first collection 2102. More particularly, the first end 2166 fifth tube length 2162 may be couplable to a second end 2152 of a fourth tube section 2150, for example, via the sixth coupling 2159. In at least one example embodiment, the fifth tube section 2160 (and more particularly, the first end 2166 of the fifth tube section 2160) may include at least a portion of the sixth coupling 2159.

The second end 2168 of the fifth tube length 2162 may be couplable to the waste bag 2170. For example, in at least one example embodiment, a portion of the second end 2158 of the fifth tube length 2162 may extend into the waste bag 2170. In at least one example embodiment, the second end 2158 of the fifth tube length 2162 may be integrally formed with the waste bag 2170. In at least one example embodiment, a coupler (not shown) may be configured to join together the second end 2158 of the fifth tube length 2162 and a port or opening of the waste bag 2170.

The fifth tube section 2160 may include a fourth clamp 2164 positioned at a point along the fifth tube length 2162 between the first and second ends 2166, 2168. Like the first, second, and third clamps 2124, 2134, 2144, the fourth clamp 2164 may be movable between a first or open position and/or a second or intermediate position and/or a third or close position. In the first position, the fourth clamp 2164 may apply no or minimal pressure to the fifth tube length 2162 and fluid flows freely therethrough. In the intermediate position, the fourth clamp 2164 may apply a closing pressure to the fifth tube length 2162 to at least partially obscure fluid flow therethrough. In the close position, the fourth clamp 2164 may apply a closing pressure to the fifth tube length 2162 to completely obscure fluid flow therethrough. The fourth clamp 2164 may be positioned at any point along the fifth tube length 2162 between the first and second ends 2166, 2168. For example, in at least one example embodiment, the fourth clamp 2164 may be disposed about halfway along the fifth tube length 2162 between the first and second ends 2166, 2168. In various other example embodiments, the fourth clamp 2164 may be disposed along the fifth tube length 2162 closer to the first end 2166 than the second end 2168. In still other example embodiments, the fourth clamp 2164 may be disposed along the fifth tube length 2162 closer to the second end 2168 than the first end 2166.

The third collection 2106 may include a sixth tube section 2180. The sixth tube section 2180 may include a sixth tube length 2182 having a first end 2186 and a second end 2188. In at least one example embodiment, the sixth tube section 2180 may be referred to as an inlet extension line. Although a single tube length is illustrated, it should be appreciated that, in various other example embodiments, the sixth tube section 2180 may be defined by two or more tube lengths or sections including one or more clamps (like the fifth clamp 2174) and one or more couplings configured, for example, to join the two or more tube lengths.

The first end 2186 of the sixth tube length 2182 may be couplable to the post-processing unit 2210. More particularly, the first end 2186 of the sixth tube length 2182 may be couplable to a second end 2214 of the post-processing unit 2210, for example, via a seventh coupling 2183. In at least one example embodiment, the sixth tube length 2182 (and more particularly, the first end 2186 of the sixth tube length 2182) may include at least a portion of the seventh coupling 2183.

The second end 2188 of the sixth tube length 2182 may be couplable to one or more other tube sections (e.g., tube sections 2220, 2230) of the apheresis systems and/or another tubing and bag set (e.g., tubing and bag set 201 as illustrated in FIG. 2) that is itself couplable to the apheresis system. For example, in at least one example embodiment, the sixth tube length 2180 (and more particularly, the second end 2188 of the sixth tube length 2182) may include at least a portion of an eighth coupling 2189 that is configured to join the sixth tube length 2182 to the one or more other tube sections (e.g., tube sections 2220, 2230) of the apheresis systems and/or another tubing and bag set (e.g., tubing and bag set 201 as illustrated in FIG. 2) that is itself couplable to the apheresis system.

The sixth tube section 2180 may include a fifth clamp 2174 positioned at a point along the sixth tube length 2182 between the first and second ends 2186, 2188. Like the first, second, third, and fourth clamps 2124, 2134, 2144, 2164, the fifth clamp 2174 may be movable between a first or open position and/or a second or intermediate position and/or a third or close position. In the first position, the fifth clamp 2174 may apply no or minimal pressure to the sixth tube length 2182 and fluid flows freely therethrough. In the intermediate position, the fifth clamp 2174 may apply a closing pressure to the sixth tube length 2182 to at least partially obscure fluid flow therethrough. In the close position, the fifth clamp 2174 may apply a closing pressure to the sixth tube length 2182 to completely obscure fluid flow therethrough. The fifth clamp 2174 may be positioned at any point along the sixth tube length 2182 between the first and second ends 2186, 2188. For example, in at least one example embodiment, the fifth clamp 2174 may be disposed about halfway along the sixth tube length 2182 between the first and second ends 2186, 2188. In other embodiments, the fifth clamp 2174 may be disposed along the sixth tube length 2182 closer to the first end 2186 than the second end 2188. In still other embodiments, the fifth clamp 2174 may be disposed along the sixth tube length 2182 closer to the second end 2188 than the first end 2186.

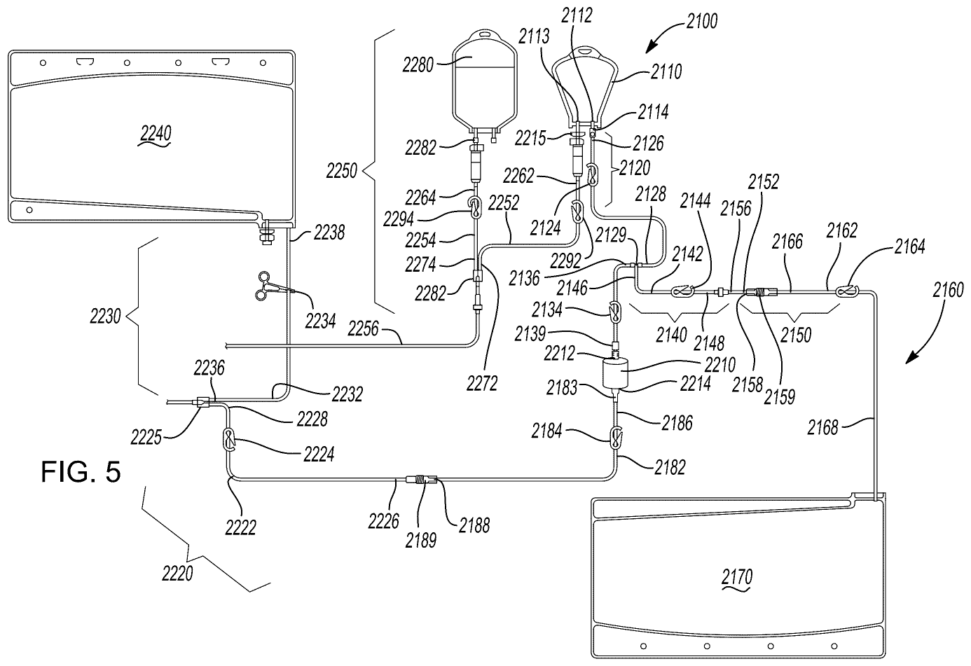

In at least one example embodiment, the tubing and bag set 2100 may include the post-processing unit. In at least one example embodiment, the tubing and bag set 2100 may include, or be in communication, with one or more other tube sections. FIG. 5 is an illustration of the example tubing and bag set 2100 as illustrated in FIG. 4, where the example tubing and bag set 2100 includes the post-processing unit 2210 and includes one or more further tube lengths 2220, 2230, 2250 for aligning the post-processing unit 2210 for use in or with apheresis devices, like the example apheresis system illustrated in FIG. 1. In at least one example embodiment, the tubing and bag set 2100 may further include, or be in communication with, a seventh tube section 2220 and/or an eighth tube section 2230 and/or a ninth tube section 2250, as illustrated in FIG. 5.

The seventh tube section 2220 may include a seventh tube length 2222 having a first end 2226 and a second end 2228. In at least one example embodiment, the seventh tube section 2220 may be referred to as a plasma divert line. Although a single tube length is illustrated, it should be appreciated that, in various other example embodiments, the seventh tube section 2220 may be defined by two or more tube lengths or sections including one or more clamps (like the sixth clamp 2224) and one or more couplings configured, for example, to join the two or more tube lengths or sections.

The first end 2226 of the seventh tube length 2220 may be couplable to the second end 2188, for example, via the eighth coupling 2189. For example, in at least one example embodiment, the seventh tube section 2220 (and more particularly, the first end 2226 of the seventh tube length 2220) may include at least a portion of the eighth coupling 2189.

The second end 2228 of the seventh tube length 2222 may be couplable to an eighth tube section 2230 for example, via a ninth coupling 2225. More particularly, the second end 2228 of the seventh tube length 2222 may be couplable to a first end 2236 of the eighth tube section 2230. In at least one example embodiment, the seventh tube length 2222 (and more particularly, the second end 2228 of the seventh tube length 2222) may include at least a portion of the ninth coupling 2225.

The seventh tube section 2220 may include a sixth clamp 2224 positioned at a point along the seventh tube length 2222 between the first and second ends 2226, 2228. Like the first, second, third, fourth, and fifth clamps 2124, 2134, 2144, 2164, 2174, the sixth clamp 2224 may be movable between a first or open position and/or a second or intermediate position and/or a third or close position. In the first position, the sixth clamp 2224 may apply no or minimal pressure to the seventh tube length 2222 and fluid flows freely therethrough. In the intermediate position, the sixth clamp 2224 may apply a closing pressure to the seventh tube length 2222 to at least partially obscure fluid flow therethrough. In the close position, the sixth clamp 2224 may apply a closing pressure to the seventh tube length 2222 to completely obscure fluid flow therethrough. The sixth clamp 2224 may be positioned at any point along the seventh tube length 2222 between the first and second ends 2226, 2228. For example, in at least one example embodiment, the sixth clamp 2224 may be disposed about halfway along the seventh tube length 2222 between the first and second ends 2226, 2228. In various other example embodiments, the sixth clamp 2224 may be disposed along the seventh tube length 2222 closer to the first end 2226 than the second end 2228. In still other example embodiments, the sixth clamp 2224 may be disposed along the seventh tube length 2222 closer to the second end 2228 than the first end 2226.

The eighth tube section 2230 may include an eighth tube length 2232 having a first end 2236 and a second end 2238. In at least one example embodiment, the eighth tube section 2230 may be referred to as a waste line. Although a single tube length is illustrated, it should be appreciated that, in various other example embodiments, the eighth tube section 2230 may be defined by two or more tube lengths or sections including one or more clamps (like the seventh clamp 2234) and/or one or more couplings configured, for example, to join together the two or more tube lengths or sections.

The first end 2236 of the eighth tube section 2230 may be culpable to the seventh tube section 2220 (and more particularly, the second end 2228 of the seventh tube section 2220), for example, via the ninth coupling 2225. In at least one example embodiment, the eighth tube section 2230 (and more particularly, the first end 2236 of the eighth tube section 2230) may include at least a portion or the ninth coupling 2225.

The second end 2238 of the eighth tube length 2232 may be couplable to a waste bag or container 2240. For example, in at least one example embodiment, a portion of the second end 2238 of the eighth tube length 2232 may extend into the bag 2240. In at least one example embodiment, the second end 2238 of the eighth tube length 2232 may be integrally formed with the bag 2240. In at least one example embodiment, a coupler (not shown) may be configured to join together the second end 2238 of the eighth tube length 2232 and a port or opening of the bag 2240.

The eighth tube section 2230 may include a seventh clamp 2234 positioned at a point along the eighth tube length 2232 between the first and second ends 2226, 2228. Like the first, second, third, fourth, fifth, and sixth clamps 2124, 2134, 2144, 2164, 2174, 2224, the seventh clamp 2234 may be movable between a first or open position and/or a second or intermediate position and/or a third or close position. In the first position, the seventh clamp 2234 may apply no or minimal pressure to the eighth tube length 2232 and fluid flows freely therethrough. In the intermediate position, the seventh clamp 2234 may apply a closing pressure to the eighth tube length 2232 to at least partially obscure fluid flow therethrough. In the close position, the seventh clamp 2234 may apply a closing pressure to the eighth tube length 2232 to completely obscure fluid flow therethrough. The seventh clamp 2234 may be positioned at any point along the eighth tube length 2232 between the first and second ends 2236, 2238. For example, in at least one example embodiment, the seventh clamp 2234 may be disposed about halfway along the eighth tube length 2232 between the first and second ends 2236, 2238. In various other example embodiments, the seventh clamp 2234 may be disposed along the eighth tube length 2232 closer to the first end 2236 than the second end 2238. In still other example embodiments, the seventh clamp 2234 may be disposed along the eighth tube length 2232 closer to the second end 2238 than the first end 2236. In at least one example embodiment, the clamp action of the seventh clamp 2234 may be obtained using, instead, clamping scissors.

The ninth tube section 2250 may include a ninth tube length 2252, a tenth tube length 2254, and/or an eleventh tube length 2256. The ninth tube length 2252 may have a first end 2262 and a second end 2272. The first end 2262 of the ninth tube length 2252 may be couplable to the second port or opening 2113 of the buffer bag 2110. In at least one example embodiment, the ninth tube length 2252 (and more particularly, the first end 2262 of the ninth tube length 2252) may include at least a portion of the second coupling 2115. Although not illustrated, it should be appreciated that, in various other example embodiments, a portion of the ninth tube length 2252 may extend into the buffer bag 2110. For example, the ninth tube length 2252 (and more particularly, the first end 2262 of the ninth tube length 2252) may include a spike that is inserted into the buffer bag 2110. The second end 2272 of the ninth tube length 2252 and also the second end 2274 of the tenth tube length 2254 may each connect to the eleventh tube length 2256. For example, an eleventh coupling 2282 may join together the ninth tube length 2254, the tenth tube length 2254, and the eleventh tube length 2256.

The ninth tube section 2250 may include an eighth clamp 2292 positioned at a point along the ninth tube length 2252 between the first and second ends 2262, 2272. Like the first, second, third, fourth, fifth, sixth, and seventh clamps 2124, 2134, 2144, 2164, 2174, 2224, 2234, the eighth clamp 2292 may be movable between a first or open position and/or a second or intermediate position and/or a third or close position. In the first position, the eighth clamp 2292 may apply no or minimal pressure to the ninth tube length 2252 and fluid flows freely therethrough. In the intermediate position, the eighth clamp 2292 may apply a closing pressure to the ninth tube length 2252 to at least partially obscure fluid flow therethrough. In the close position, the eighth clamp 2292 may apply a closing pressure to the ninth tube length 2252 to completely obscure fluid flow therethrough. The eighth clamp 2292 may be positioned at any point along the ninth tube length 2252 between the first and second ends 2262, 2272. For example, in at least one example embodiment, the eighth clamp 2292 may be disposed about halfway along the ninth tube length 2252 between the first and second ends 2262, 2272. In various other example embodiments, the eighth clamp 2292 may be disposed along the ninth tube length 2252 closer to the first end 2262 than the second end 2272. In still other example embodiments, the eighth clamp 2292 may be disposed along the ninth tube length 2252 closer to the second end 2272 than the first end 2262. In at least one example embodiment, the clamp action of the eighth clamp 2292 may be obtained using, instead, clamping scissors.

The tenth tube length 2254 may have a first end 2264 and a second end 2274. The first end 2264 of the tenth tube length 2254 may be couplable to a bag 2280. In at least one example embodiment, the bag 2280 may be a vent bag. In at least one example embodiment, the tenth tube length 2254 (and more particularly, the first end 2264 of the tenth tube length 2254) may include at least a portion of a tenth coupling 2282 the joins together the bag 2280 and the tenth tube length 2254. Although not illustrated, it should be appreciated that, in various other example embodiments, a portion of the tenth tube length 2254 may extend into the bag 2280. For example, the tenth tube length 2254 (and more particularly, the first end 2264 of the tenth tube length 2254) may include a spike that is inserted into the bag 2280.

The ninth tube section 2250 may include a ninth clamp 2294 positioned at a point along the tenth tube length 2254 between the first and second ends 2264, 2274. Like the first, second, third, fourth, fifth, sixth, seventh, and eight clamps 2124, 2134, 2144, 2164, 2174, 2224, 2234, 2292, the ninth clamp 2294 may be movable between a first or open position and/or a second or intermediate position and/or a third or close position. In the first position, the ninth clamp 2294 may apply no or minimal pressure to the tenth tube length 2254 and fluid flows freely therethrough. In the intermediate position, the ninth clamp 2294 may apply a closing pressure to the tenth tube length 2254 to at least partially obscure fluid flow therethrough. In the close position, the ninth clamp 2294 may apply a closing pressure to the tenth tube length 2254 to completely obscure fluid flow therethrough. The ninth clamp 2294 may be positioned at any point along the tenth tube length 2254 between the first and second ends 2264, 2274. For example, in at least one example embodiment, the ninth clamp 2294 may be disposed about halfway along the tenth tube length 2254 between the first and second ends 2264, 2274. In various other example embodiments, the ninth clamp 2294 may be disposed along the tenth tube length 2254 closer to the first end 2264 than the second end 2274. In still other example embodiments, the ninth clamp 2294 may be disposed along the tenth tube length 2254 closer to the second end 2274 than the first end 2264.

In at least one example embodiment, one or more of the different clamps 2124, 2134, 2144, 2164, 2174, 2234, 2292, 2294 may have one or more different colors to help an operator identify appropriate event sequences. In at least one example embodiment, the clamp action of any of the one or more clamps 2124, 2134, 2144, 2164, 2174, 2234, 2292, 2294 may be obtained using, instead, clamping scissors.

In various aspects, the present disclosure provides methods of using tubing and bag sets including post-processing units (like the tubing and bag sets 2100 illustrated in FIGS. 4 and 5) for use in or with medical systems (like the example apheresis system illustrated in FIG. 1). The methods may generally include aligning the selected tubing and bag set with or within the medical systems and/or the donor or the subject or the patient or the source. In at least one example embodiment, for example, when the post-processing units of the selected tubing and bag set includes a filter, the methods may include one or more flushing steps to remove or limit any clogs or blockages in the post-processing units.

In at least one example embodiment, methods of using tubing and bag sets including post-processing units (like the tubing and bag sets 2100 illustrated in FIGS. 4 and 5) in or with medical systems (like the example apheresis system illustrated in FIG. 1) may include introducing a blood component (e.g., plasma) to the post-processing unit (e.g., post-processing unit 2210 as illustrated in FIG. 5) from a tube length couplable to a donor or a subject or a patient or a source (e.g., tube length 2220 as illustrated in FIG. 5). After the post processing, the methods may include re-infusing to the donor or the subject or the patient or the source one or more other blood components (e.g., red blood cells and/or white blood cells and/or platelets) via another tube length (e.g., tube length 2256 as illustrated in FIG. 5). In at least one example embodiment, the one or more other blood components (e.g., red blood cells and/or white blood cells and/or platelets) may be recirculated to the donor or the subject or the patient or the source via a reservoir (e.g., reservoir 248 as illustrated in FIG. 2).

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A medical system comprising:
a separation device configured to separate a material received from a source into two or more components;
a post-processing unit in fluid communication with the separation device and configured to isolate at least one of the two or more components; and
a controller configured to manage operation of the separation device to separate the material received from the source into the two or more components, to cause the two or more components to move to the post-processing unit, and to return the isolated at least one of the two or more components to the separation device.

2. The medical system of claim 1, wherein the medical system further includes:
a tube establishing the fluid communication between the separation device and the post-processing unit.

3. The medical system of claim 2, wherein the fluid communication between the separation device and the post-processing unit is a first fluid communication between the separation device and the post-processing unit, and the medical system further includes:
a second fluid communication between the post-processing unit and the separation device to return the isolated at least one of the two or more components to the separation device.

4. The medical system of claim 3, wherein the tube is a first tube and the medical system further includes:
a second tube establishing the second fluid communication between the post-processing unit and the separation device to return the isolated at least one of the two or more components to the separation device.

5. The medical system of claim 4, wherein the first and second tubes are provided as a connectivity set.

6. The medical system of claim 4, wherein the medical system further includes:
a third tube establishing fluid communication between the separation device and the source.

7. The medical system of claim 6, wherein the first, second, and third tubes are provided as a connectivity set.

8. The medical system of claim 6, wherein the second and the third tubes are provided as a connectivity set.

9. The medical system of claim 1, wherein the post-processing unit includes a column.

10. The medical system of claim 1, wherein the post-processing unit includes a filter.

11. A therapeutic system comprising:
a flow path fluidically connecting a separation device and a post-processing device, the separation device configured to separate a material received from a source into two or more components, and the post-processing device configured to isolate at least one of the two or more components.

12. The therapeutic system of claim 11, wherein the flow path is a first flow path, and the therapeutic system further includes:
a second flow path fluidically connecting the separation device and the post-processing device to return the isolated at least one of the two or more components to the separation device.

13. The therapeutic system of claim 12, wherein the first flow path and the second flow path are provided as a connectivity set.

14. The therapeutic system of claim 12, wherein the therapeutic system further includes:
a third flow path fluidically connecting the separation device and the source.

15. The therapeutic system of claim 14, wherein the first, second, and third flow paths are provided as a connectivity set.

16. The therapeutic system of claim 14, wherein the second and the third flow paths are provided as a connectivity set.

17. The therapeutic system of claim 11, wherein the post-processing device includes a column.

18. The therapeutic system of claim 11, wherein the post-processing device includes a filter.

19. A connectivity set for connecting a separation device and a post-processing unit, the connectivity set comprising:
a first tube collection including a first tube length having a first end and a second end, the first end of the first tube length being couplable to the separation device, the second end of the first tube length being couplable to the post-processing unit; and
a second tube collection including a second tube length having a first end and a second end, the first end of the second tube length being couplable to the post-processing unit and the second end of the second tube length being couplable to the separation device,
wherein the separation device is configured to separate a material received from a source into two or more components, and the post-processing unit is configured to isolate at least one of the two or more components.

20. The connectivity set of claim 19, wherein the first tube collections includes one or more first clamps.

21. The connectivity set of claim 20, wherein the second tube collection includes one or more second clamps.

22. The connectivity set of claim 21, wherein the one or more first clamps are a first color and the one or more second clamps are a second color, the second color being different from the first color.

23. The connectivity set of claim 19, wherein the connectivity set further includes:
a third tube collection including a third tube length couplable to the separation device and the source.

24. The connectivity set of claim 22, wherein the third tube collection includes one or more third clamps.

25. A connectivity set for connecting a separation device and a post-processing unit, the connectivity set comprising:
a first tube length having a first end and a second end, the first end of the first tube length being couplable to a first bag;
a second tube length having a first end and a second end, the first end of the second tube length being couplable to the second end of the first tube length, and the second end of the second tube length being couplable to a first end of the post-processing unit;
a third tube length having a first end and a second end, the first end of the third tube length being couplable to the second end of the first tube length;
a fourth tube length having a first end and a second end, the first end of the fourth tube length being couplable to the second end of the third tube length, and the second end of the fourth tube length being couplable to a second bag; and
a fifth tube length having a first end and a second end, the first end of the fifth tube length being couplable to a second end of the post-processing unit, and the second end of the fifth tube length being couplable to one or more other components of the separation device.

26. The connectivity set of claim 25, wherein the first tube length, the second tube length, and the third tube length are joined and provided as a first distinct tube collection.

27. The connectivity set of claim 26, wherein the fourth tube is provided as a second distinct tube collection.

28. The connectivity set of claim 27, wherein the fifth tube is provided as a third distinct tube collection.

29. The connectivity set of claim 28, wherein at least one of the first tube length, the second tube length, the third tube length, the fourth tube length, and the fifth tube length includes one or more clamps.
